(19) United States
(12) Patent Application Publication
(10) Pub. No.: US 2026/0279550 A1
(43) Pub. Date: Sep. 17, 2026

(54) ENHANCED REAL-TIME PROCESSING OF LIVE X-RAY IMAGE STREAMS USING MACHINE LEARNING AND ARTIPICIAL INTELLIGENCE FOR OPTIMIZATION OF PATIENT OUTCOMES IN EMBOLIZATION PROCEDURES

(71) Applicant: Emerald Neurovascular, Hunts Point, WA (US)

(72) Inventors: William Monteith, Seattle, WA (US); Stephen Monteith, Seattle, WA (US)

(73) Assignee: Emerald Neurovascular, Hunts Point, WA (US)

(21) Appl. No.: 19/566,176

(22) Filed: Mar. 13, 2026

Related U.S. Application Data
(60) Provisional application No. 63/771,829, filed on Mar. 14, 2025.

(51) Int. Cl. G16H 30/40; G06T 7/00

(52) U.S. Cl. CPC G16H 30/40; G06T 7/0012; G06T 2207/10116

(57) ABSTRACT

Hardware and software improvements support Real-time image processing system (RTIPS) algorithm effective for detection of intrusion into protectable zones during, for example the special example of the onset of dangerous embolization agent flow/movement during Middle Meningeal Artery Embolization; along with analogous data set harvesting during Embolization of arterio-venous malformations of the brain; Embolization of arterio-venous fistulae of the brain and its coverings (dura); Cerebral Aneurysm embolization; Neuro-interventional embolization procedures of the head, neck and central nervous system; Injection of cement into bony structures (during kyphoplasty or vertebroplasty); Hardware (screw) placement during spinal instrumentation procedures; Arterial embolization procedures of organs or lesions in the human body; and, Venous embolization procedures of organs or lesion in the human body.

### CROSS-REFERENCE TO RELATED CASES

Priority is claimed to U.S. Provisional Ser. No. 63/771,829 filed Mar. 14, 2025 by the instant assignee, Emerald Neurovascular.

### BACKGROUND OF THE INVENTIONS

Delivery of liquid embolic to the neurovascular system is fraught with issues. Prominent among them is having them flow or leak into places where they can have major negative brain impacts. A modicum of general neurovascular procedural background is believed to cast the scope of the present inventions from this one specifically denoted example to the myriad of related and treatable regions, disease states and procedures of the present disclosures and inventions, it is proposed.

It is further respectfully submitted that the MMA or middle meningeal artery (arteria meningea media) is typically the third branch of the first portion of the maxillary artery and an important region wherein embolization events are often required. Observation of how the MMA Embolization procedure endovascularly delivered can be enhanced and made more accurate is paradigmatic of how the present invention can apply to related procedures, or those with commonalities.

The neurovascular system comprises, inter alia, a complex network of blood vessels and arteries which supply the brain and spinal cord with oxygen and nutrients. Conversely, venous portions of the system serve to drain blood and waste products from the brain. For example, the great vein of Galen drains the anterior and central regions of the brain, emptying into the dural sinuses, thereby preventing a buildup of pressure in the blood vessels.

The blood supply to the brain is via two main pairs of arteries: the carotid arteries and vertebral arteries. The carotid arteries are located in the front of the neck and mainly supply the front and middle parts of the brain, by dividing into anterior cerebral and middle cerebral arteries on each side.

The front part of the brain, mainly the frontal lobe, is responsible for thinking, planning, decision-making, problem-solving, controlling behavior and emotions, and movement. It also plays a role in speech. The middle part of the brain, which includes parts of the parietal lobe and deeper structures, helps with processing touch, pain, temperature, and spatial awareness. It also plays a role in coordinating movement and integrating sensory information from different parts of the body.

The vascular system of the back of the brain includes the vertebral arteries, basilar artery, and other arteries that supply blood to the cerebellum, brainstem, and occipital lobes.

Regarding vertebral arteries, two vertebral arteries branch from the subclavian arteries in the neck and run generally along with the spinal column into the skull. The basilar artery, essentially the main artery that supplies blood to the back of the brain (comprising the posterior cerebral circulation), originates where the vertebral arteries join near the brainstem, and effectively supplies blood to the brainstem, cerebellum, and occipital lobes. The Circle of Willis includes a loop of blood vessels near the bottom of the brain that connects major arteries, and provides a redundant function, ensuring that blood can flow to the affected part of the brain, should there be an ischemic issue with one of the main arteries.

The back of the brain is integral to proper physiological function, and controls many vital functions, including breathing, heart rate, swallowing, vision, movement, and balance.

When the arteries supplying blood to the brain are blocked due to a thrombus (a blood clot) the brain cells in that area start to die within minutes. This condition is referred to as the ischemic brain stroke and is a medical emergency. The effects of the stroke depend on which artery is blocked and the part of the brain affected. Ischemic strokes are the most common type of stroke and need fast treatment to reduce brain damage. If blockage involves the venous system of the brain, then blood builds up, causing swelling, pressure, and sometimes bleeding in the brain. This can lead to headaches, seizures, vision problems, weakness and in severe cases death. Treatment involves administration of thrombolytics (blood thinners) to eliminate the blockage and restore blood flow.

Specialized catheters (microcatheters) utilizing dimensional miniaturization of a typical microcatheter diameter have been suggested, and which are designed for treatment of neurovascular conditions including cerebral aneurysms, ischemic strokes, and other deleterious conditions that affect the vessels of the brain. Such miniaturization is advisable inasmuch as such microcatheters ofttimes need to navigate tortuous and delicate blood vessels of the head or neck without inflicting unintended damage thereto.

While specialized catheters are primarily used in neurointerventional procedures, for example treatment of brain aneurysms, where different sizes are needed to access and navigate various blood vessels depending on the patient's anatomy. They have typically been provided optionally with a generally soft, rounded tip for smooth navigation and a flexible distal shaft to maneuver through complex and often tortuous blood vessel curves, while maintaining a stable proximal shaft for device delivery to a target intravascular location.

While the noted conventional approaches to treatment of vessels in the neurovascular system have proved effective to a degree, they appear to have failed to address all considerations, including reliable trackability/deliverability to a treatment site, proximal support and distal navigation and reach, and the option for simultaneous performance of plural treatment options.

It would therefore be desirable to offer a real-time x-ray based imaging and AI enhanced guidance system which is particularly well suited to interventionally diagnose and/or treat neurovascular anomalies, and which addresses the drawbacks of the current state of the art.

### SUMMARY OF THE INVENTIONS

Hardware and software improvements support at least a real-time image processing system (RTIPS) algorithm effective for detection of intrusions into protectable zones during, for example the specific example of the onset of dangerous embolization agent flow/movement during Middle Meningeal Artery Embolization; along with analogous data set harvesting during Embolization of arterio-venous malformations of the brain; Embolization of arterio-venous fistulae of the brain and its coverings (dura); Cerebral Aneurysm embolization; Neuro-interventional embolization procedures of the head, neck and central nervous system; Injection of cement into bony structures (during kyphoplasty or vertebroplasty); Hardware (screw) placement during spinal instrumentation procedures. Arterial embolization procedures of organs or lesions in the human body; and, venous embolization procedures of organs or lesion in the human body.

In accordance with these and other objects of the invention, the present inventors have discovered that a proposed real-time processing system yields significant reduction in the numbers of sustained harmful embolization agent flow events and improve patient procedure outcomes.

According to embodiments, the system will include continuous machine learning and artificial intelligence (AI) capabilities, at the local procedure room level, which record and analyze all procedure activity to inform continuous enhancement of the system capability and accuracy for individual surgeons and teams of surgeons.

It is also proposed that the machine learning and AI capabilities will be generalized on a worldwide scale, using an internet cloud-based Machine Leaming and AI Hub. This receives its procedure data flows from all participating local operating/procedure rooms and distribute updated programming of the local systems as the machine learning and AI databases incorporate expanding procedure experience.

According to embodiments, used as a tool by users/operators AI works well with the data sets harvested such that the continuous system programming improvement enabled by the real-time machine learning and AI features will lead to fully automated generation of the embolization agent danger zones with accuracy matching or exceeding what can currently be achieved with solely human scrutiny of the live scanner image streams.

These and other features and advantages are described with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a schematic flow-chart type of illustration summarizing key features of the system supporting RTIP software via a schematic flow diagram to outline the process of the inventive systems according to embodiments of the present inventions;

FIG. 2 shows an exemplary inventive synthetic environment for cloud-based machine learning and AI Hub configurations according to the teachings of the present inventions.

FIG. 3 depicts partial perspective views of an exemplary live x-ray scanner video image stream with overlaid transparent danger zone mark-up layers according to embodiments of the present inventions;

FIG. 4 is a schematic flow chart showing the process of the inventive system according to embodiments of the present inventions;

FIG. 5 schematically illustrates an orthogonal crosshair image processing cursor;

FIG. 6 is a schematic and exemplary cursor assembly for implementing inventions of the present disclosures; and

FIG. 7 is a schematic and exemplary process using subject cursor assemblies for implementing inventions of the present disclosures for open danger zone segments.

FIG. 8 is a schematic and exemplary process using subject cursor assemblies for implementing inventions of the present disclosures for closed danger zone segments.

### DETAILED DESCRIPTION OF THE INVENTION

The present inventors have discovered that a real-time processing system yields significant reductions in the numbers of sustained harmful embolization agent flow events and can improve patient procedure outcomes. This is effectuated by delineation of zones of danger into which, for example, liquid embolic, are not intended to pass. Likewise, those of skill in the art can extrapolate to numerous other boundary condition driven procedures and readily apply the objects of the present invention to better management of the same, it is respectfully proposed.

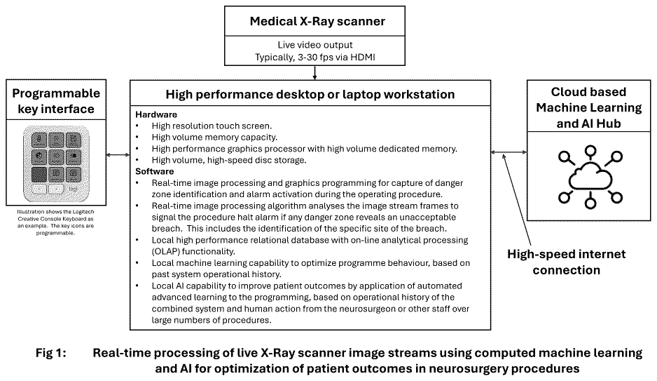

As shown in and referring to FIG. 1, as disclosed the system is improved by using continuous machine learning and artificial intelligence (AI) capabilities, at the local procedure room level, which record and analyze all procedure activity to inform continuous enhancement of the system capability and accuracy for individual surgeons and teams of surgeons. For ease of explanation, details known to artisans are omitted for ease and clarity in defining the instant inventions. As shown in FIG. 1, a medical x-ray scanner is a typical medical scanner with a live, 3-30 frames per second, image output stream via an HDMI port. The image streams will usually have several image panels in each frame. These show views of the target site, some of which have been subject to live pre-processing at source in the scanner. An example is the production of an initial reference scan image which is fixed at the commencement of a procedure and this image color definition may then be subtracted from live scan stream color data to improve observation of the progress of the embolization agent once it emerges from the nozzle of the delivery catheter in the artery or vein. The scanner output is connected to the appropriate HDMI input of the high-performance computer.

A high-performance desktop or laptop workstation provides the primary observation and control interface for the neurosurgeon and/or other operating staff. Standard hardware, such as known to those skilled in the art is comprised of at least a high-resolution touch screen, high volume memory capacity, a high-performance graphics processor with high volume dedicated memory and high-speed disc storage.

Software, as defined herein and claimed below is a real-time image processing and graphics programming (RTIP) system for the capture of danger zone definition and alarm activation and display during the operating procedure. The real-time image processing algorithm analyses the image stream frames to signal the procedure halt alarm if any danger zone reveals an unacceptable breach. This includes the identification of the specific sites of the breaches.

A local high performance relational database with on-line analytical processing (OLAP) functionality facilitates exploration of the dependency of dangerous embolization agent rows on known or potential factors of influence-such as patient age, patient medical conditions or experience levels of operating staff. The operating dangerous embolization agent flow data can be stored as the dependent variable in the OLAP cube model and the influence parameters data will be stored in the OLAP cube dimensions, as available operational data is accumulated, it can inform the improvement of the local machine learning capability to optimize the computer program behavior.

Local AI capability to improve patient outcomes by application of automated advanced learning to the programming, based on operational history of the combined system and human action from the neurosurgeon or other staff over large numbers of procedures.

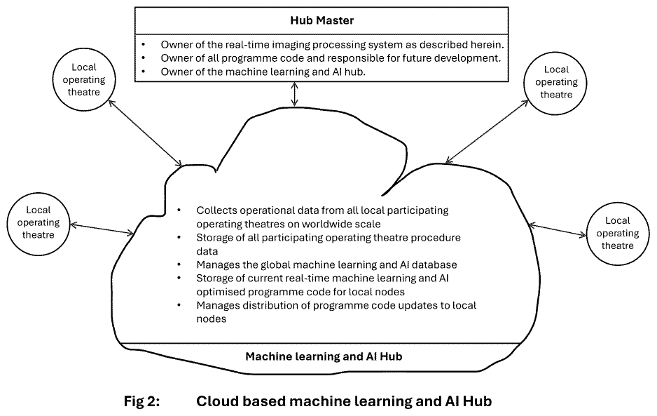

FIG. 1 and FIG. 2 specifically disclose and claim that the machine learning and AI capabilities are generalized on a worldwide scale, using an internet cloud-based Machine Leaming and AI Hub. These improvements enable the system to receive its procedure data flows from all participating local operating/procedure rooms and distribute updated programming of the local systems as the machine learning and Al databases incorporate expanding procedure experience. Likewise, it is posited that the continuous system programming improvement enabled by the real-time machine learning and AI features will lead to fully automated generation of the embolization agent danger zones with accuracy matching or exceeding what can currently be achieved with human scrutiny of the live scanner image streams.

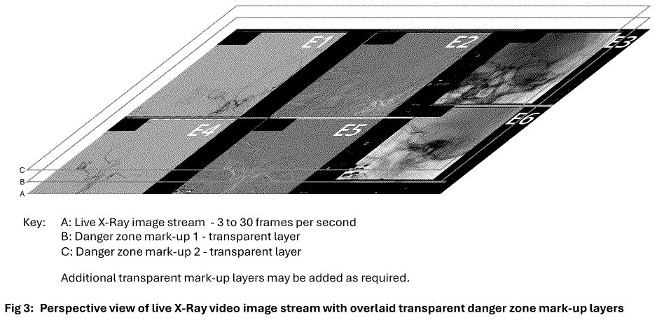

FIG. 2 through FIG. 5 show the system as arrayed to be controlled by at least a user including via the programmable keyboard and related interface tools. FIG. 3 illustrates the Real-time image processing system (RTIPS) and live X-Ray scanner video stream with overlaid transparent danger zone mark-up layers. This partial perspective plan view shows a perspective view of the RTIPS layered windows or layered window objects on the workstation touch screen.

FIG. 3 shows the perspective view of the live X-Ray scanner video image stream with overlaid transparent danger zone mark-up layers. The Key explains that
A is the Live X-Ray image stream—3 to 30 frames per second.
B is the Danger zone mark-up 1—transparent layer.
C: is the Danger zone mark-up 2 transparent layer and that additional transparent mark-up layers may be added as required.

Layer A displays the live video stream from the X-Ray scanner. This example image frame is from a middle meningeal artery embolization procedure. This layer is at the bottom of the layer stack. Transparent danger zone mark-up Layer B—when this layer has focus it is the target for screen touch pen (or mouse) strokes made by the operator. The layer boundary is color coded and the pen/mouse ink color matches the chosen layer boundary color. The operator uses the pen/mouse to draw danger zone segments on the layer. All likely danger zones are identified by inspection of the live image stream in layer A, prior to the introduction of the embolization agent.

All pen/mouse stroke data is recorded to memory and disk (as in figures). The pixels in danger zone segments in the layer are used by the RTIPS to identify the corresponding pixels to be examined in the live X-Ray video frames in layer A.

The RTIPS algorithm processes the live image stream in layer A-but does not modify the live stream data. The raw image stream frames are saved to memory and disk. The color coding of the layer boundary and the pen/mouse ink provides a convenient mechanism for the operator to group danger zones that are deemed to be of a specific type or that point to locations with similar risk of dangerous embolization agent flow. The layers may be hidden and reinstated to their registered locations on the workstation screen to facilitate close inspection of the underlying video and adjustment of the segment positioning, if necessary. Any danger zone segment may be removed by selecting an erase function and using the pen/mouse to touch the segment. Additional transparent danger zone mark-up layers, in the form of additional color coded mark-up layers, such as layer C may be added as required. When multiple mark-up layers are used, all danger zone segments on all transparent layers will be processed by the RTIPS even if the layers are hidden.

According to preferred embodiments, a workstation monitor view of live X-Ray scanner video image stream works for a typical workstation monitor view of a live X-Ray scanner video image stream from a Middle Meningeal Embolization procedure, displayed in the RTIPS layer. In FIG. 3, the workstation monitor view with transparent mark-up layer B has its danger zone segments superimposed over layer A. Likewise, a monitor view of the live X-Ray video image stream with overlaid transparent danger zone mark-up layer 1 works as does the workstation monitor view with transparent mark-up layer C with its danger zone segments superimposed over layer A. The system similarly embraces a monitor view of the live X-Ray video image stream with overlaid transparent danger zone mark-up layer 2 and the workstation monitor view with both mark-up layers B and C, with their danger zone segments, superimposed over the live image stream layer A, along with the live X-Ray video image stream with overlaid transparent danger zone mark-up layers 1 and 2. It is important to note that the stack order of the transparent layers is not significant for the RTIPS algorithm.

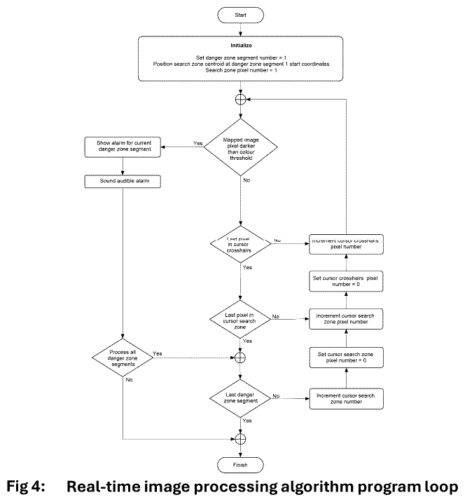

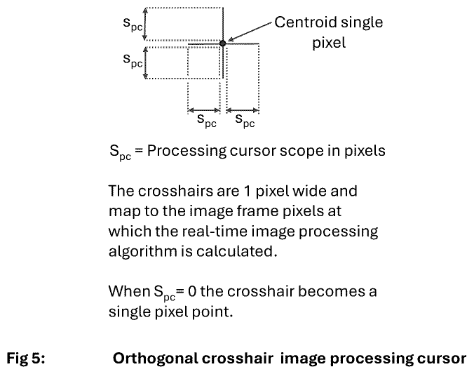

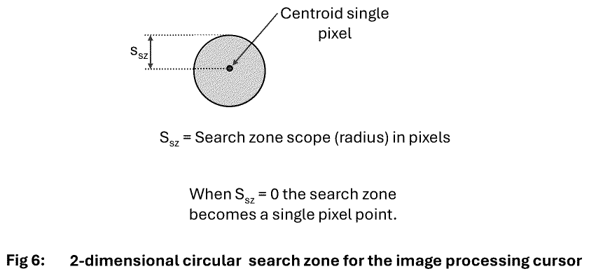

FIG. 4 FIG. 5 and FIG. 6 present the basis of the Real-time Image Processing System (RTIPS) algorithm for detection of the onset of dangerous embolization agent flow/movement according to the present invention, as described and claimed below.

FIG. 5 and FIG. 6 show an orthogonal crosshair image processing cursor, and 2-dimensional circular search zone for the image processing as known to those skilled in the art, an orthogonal crosshair image is generated by a processing cursor using a centroid single pixel approach. A 2-dimensional circular search zone for the image processing cursor is defined by the centroid single pixel working in concert with the processing cursor scope in pixels. The crosshairs are 1 pixel wide and map to the image frame pixels at which the real-time image processing algorithm is calculated. The search zone scope, S_{pc} defines a circular search area radius in pixels. When S_{pc}=0 the search zone becomes a single pixel point. The cursor arms are 1 pixel in thickness, the cursor scope and Sₚ is defined as the number of pixels that the cursor arms extend vertically and horizontally beyond their centroid pixel. For example, if Sₚ=3, the horizontal cursor arm will be 7 pixels wide with its centroid at pixel 4 and the vertical arm will be 7 pixels high with the centroid pixel at pixel 4.

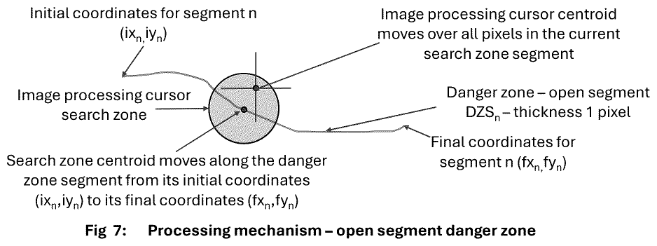

The image processing cursor is used by RTIPS to specify the pixel location coordinates that will be mapped from the danger zone mark-up layers to the underlying live image stream layer as the processing algorithm operates on the 2-dimensional circular search zones for the image processing cursor. FIG. 7 shows the search circular search zone with a scope (radius) of S_{sz} pixels. The image processing algorithm will move the processing cursor centroid over all pixels specified on and within the search zone circumference.

The image processing algorithm will perform its analysis on the underlying image for every pixel in the processing cursor orthogonal crosshair arms as it moves the cursor centroid over all pixels specified by the cursor search zone. The processing then moves the search zone centroid over all pixels specified in all the specified danger zone segments marked up in all the mark-up layers. The operator can choose at least one of:
1. Having the RTIPS complete the processing of the entire set of marked up danger zone segments to identify those with dangerous embolization agent flows on the workstation screen with appropriate visual/audible alarms.
2. Exit processing when the first dangerous embolization agent flow is identified. Thus, the operator decides which action to take when 1 or more dangerous embolization agent flows/movement have been detected.

FIG. 7 shows an example of how this works with open segment danger zones. Here, an open segment danger zone tis added to a transparent mark-up layer. Artisans understand such a processing mechanism, where the open segment danger zone is based on initial and final coordinates and the segment thickness is 1 pixel. For segment DZSₙ, the image processing cursor centroid moves over all pixels in the current danger zone segment from the initial pixel coordinates (iXₙ, iYₙ) to the final coordinates (fXₙ, fYₙ). For each DSZₙ, defined in the mark-up layers, the processing algorithm is performed using the underlying live image stream frame data for all pixels in the segment. At each position of the image processing cursor centroid, the processing algorithm is performed for all pixels mapped from the mark-up layer to the live image as specified in both the vertical and horizontal arms of the orthogonal crosshairs.

The underlying image stream data is not modified since the danger zones are defined in the mark-up layers and provide the coordinate mapping to position the image processing cursor to apply the processing algorithm at the corresponding coordinates in the underlying image data frame, so its image data is always in read-only access.

In summary, an open segment is 1 pixel in thickness and added to a transparent mark-up layer, prior to the introduction of an embolization agent. The segment is an ad-hoc shape which has a single start point and a single end point. It does not enclose a 2-dimensional area. User specifies the movement trajectory of the centroid of the image processing cursor for mapping to the underlying live image (layer A). FIG. 7 shows an example position of the circular search zone which specifies the precise pixels that will undergo the processing algorithm search for detection of dangerous embolization agent flow. The underlying image stream data is not modified. The operator can add as many open segments as required, using experience of the operating procedure and reliable assessment of the scanner image streams.

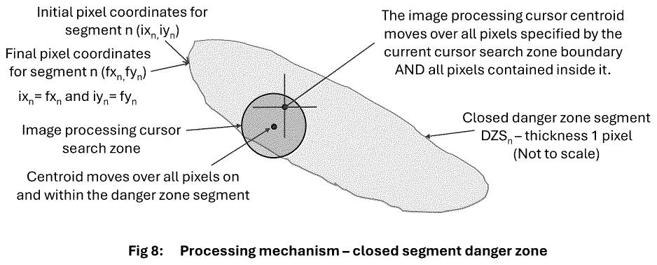

Likewise, it is proposed that Artisans can use the present invention to construct closed segment danger zones as shown in FIG. 8.

For each DSZ defined in the mark-up layers, the processing algorithm is performed using the underlying live image stream frame data for all pixels mapped from the image processing cursor as it moves along the closed danger zone search boundary and over all pixels contained within it. The underlying image stream data is not modified since the danger zones are defined in the mark-up layers and provide the coordinate mapping to position the image processing cursor to apply the processing algorithm at the corresponding coordinates in the underlying image data frame, so its image data is always in read-only access.

For scope Spc>0, the processing algorithm will include image pixels that lie outside the danger zone boundary but within the areas of the image processing cursor. A closed segment is 1 pixel in thickness and added to a transparent mark-up layer, prior to the introduction of embolization agent.

The closed segment is an ad-hoc shape which has its start and end points on the same pixel coordinates. The closed segment encloses a 2-dimensional area. It specifies the movement area for the centroid of the image processing cursor for mapping to the underlying live image (layer A). All pixels on the boundary and those enclosed inside it will map to the underlying image pixels that will undergo the processing algorithm search for detection of dangerous embolization agent flow.

FIG. 8 shows an example position of the circular search zone which specifies the precise pixels that will undergo the processing algorithm search for detection of dangerous embolization agent flow. When the search zone approaches the segment boundary some pixels outside the danger zone segment, but within the current cursor search zone, will be included in the algorithm processing. The underlying image stream data is not modified. The operator can add as many closed segments as required, using experience of the operating procedure and reliable assessment of the scanner image streams.

FIG. 4 shows the main features of the proposed Real-time Image Processing System (RTIPS) algorithm. The algorithm is processed once per frame of the live X-Ray scanner data stream. The time available for each processing cycle is therefore determined by the data stream frame rate. For a frame rate of 3 fips the available processing time is 333 ms and for a frame rate 30 fps the available processing time will be 33 ms. This imposes an important performance target for general specifications of the RTIPS workstation as discussed. The use of dedicated graphics processing and graphics memory and high CPU clock speeds is critical. The image processing orthogonal crosshairs cursor combined with the cursor search zone aims to detect dangerous embolization flows at high speed and with high reliability by minimizing the number of image pixels that participate in the search process. Likewise, the use of danger zone mark-up to pre-determine vulnerable locations in the image frame limits the number of pixels to be processed to a very small fraction of the full frame and yields a high probability of reliable detection of dangerous embolization agent flow within the processing time available.

The Real-time Image processing program loop is summarized in FIG. 4 which shows the program loop flowchart. The loop iterates on
danger zone segments
danger zone segment pixels
image processing cursor search zone pixels
and image processing cursor pixels.

The flowchart reveals the precise order of the iterations which is chosen to maximize the probability of reliable detection of dangerous embolization agent flows with adequate CPU performance for full loop execution in the time between 2 consecutive input X-Ray scanner image stream frames.

The following control parameters would normally be set by the neurosurgeon or other staff before the introduction of the embolization agent, although RTIPS would use a default parameter set if it is not manually adjusted:
1. The processing cursor scope in pixels.
2. Sp_{c}—the cursor search zone scope (radius) in pixels.
3. The color threshold for the processing algorithm. The algorithm assesses the color information for each mapped processed pixel in the current image frame and if the color is darker than the color threshold, RTIPS shall trigger the alarms to confirm a dangerous embolization agent flow has been detected.
4. The threshold could be an absolute color value or a delta value calculated as a difference in the current pixel color from its value in the immediately previous frame or by averaging from more that one previous frame.
5. The Process all danger zone segments parameter (On or Off) can be used by the operator to force the processing of all danger zone segments or exit the program loop to as soon as the first dangerous embolization agent flow is detected. When this parameter is ON multiple alarms could be triggered in the same processing cycle. All control parameter values will be stored by RTIPS. RTIPS also records all timing events for alarms triggered.

The RTIPS invention also provides for an optional retrospective mark-up layer (RMUL), at the top of the layer stack, which can be used by the neurosurgeon to add post-procedure information about how the system has performed during the entire embolization agent delivery—after the procedure has finished—either to a successful conclusion or by termination from dangerous embolization flow detection. The RMUL data is added with touch pen or mouse strokes (such as ✓ or X or XX) in the same way as for danger zone segment mark-up. Information captured could include:
1. Confirmation of which danger zone segments correctly triggered dangerous embolization agent flow alarms (✓).
2. Confirmation of which danger zone segments triggered false dangerous embolization agent flow alarms (X).
3. Confirmation of danger zone segments that did not trigger an alarm when they should have (XX).
4. The RMUL data will be stored by RTIPS.
5. As shown also in FIG. 1 and FIG. 2-3. Both AI and Machine learning training data would be stored at the local level and ultimately be sent to the cloud-based Machine Learning and AI Hub.

RTIPS will store all relevant data as discussed above for all procedures. This data expands as the number of procedures increases.

In addition, the local systems theatres/hubs capture patient information that, in the opinion of the user/operator, could have an influence on potential procedure outcomes. All of this information and protocols to transfer it will be stored in the multi-dimensional OLAP cube at the local level. This will be used to inform the machine learning and AI models to continuously optimize and update the detection of dangerous embolization program code base either:
1. When RTIPS starts at the beginning of a procedure,
Or
2. When RTIPS is shut down at the end of a procedure.

In FIG. 2 AI at the local level/hub and operating theatre is described and claimed below. As the volume of operational data grows, AI modelling is understood to be deployed at the local level and the insights gained utilized to achieve continuous, combined enhancement in the RTIPS.

While several embodiments of the present disclosure are described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that. Within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention.

Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well—known structures, materials, and so forth. In other instances, well—known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method.

Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method.

Additionally, the order in which a particular method occurs may or may not strictly adhere to the order of the corresponding steps or methodologies shown.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

While the machine-readable medium can in an exemplary embodiment be a single medium, the term “machine-readable medium” should be taken to include a single medium or multiple media (e. g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (S.D.), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e. g., EPROM, EEPROM, solid state drive (S.D.), and flash memory devices e.g., SD, micro SD, SDXC, SDIO, SDHC cards); magnetic disks, (e.g., internal hard disks or removable disks); and optical disks (e.g., CD and DVD disks).

Accordingly, the present invention is not limited to that precisely as shown and described.

Various preferred embodiments are described herein with references to the drawings in which merely illustrative views are offered for consideration, whereby:

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

What is claimed is:

1. An accuracy improved system for combining hardware and software elements to define surgical boundary conditions, such as embolization danger zones, to facilitate better management of endovascular procedures, inter alia, comprised of, in combination: an x-ray scanner; a computer workstation (desktop/laptop); a programmable key interface; an orthogonal crosshair image processing cursor set; hardware and software with on-line analytical processing (OLAP) functionality and a real-time Image processing algorithm system (RTIPS) with cloud-based and local hub linkages.

2. The accuracy improved system of claim 1, comprised of lower incidences of false positives and minimized artifacts, along with elimination of acritical notifications providing optimized resultory data sets for optimized procedural outcomes.

3. The accuracy improved system of claim 2, further comprised of real-time X-ray image processing in multiple transparent layers.

4. The accuracy improved system of claim 3, the multiple layers comprised of at least about two to three disposed in overlapped fashion; wherein a stack order of the transparent layers is not significant for the subject RTIPS algorithm.

5. The accuracy improved system of claim 4, further comprised of settings whereby the operator the operator decides which action to take when 1 or more surgical boundary conditions, such as dangerous embolization agent flows/movement have been detected by choosing at least one of:
i. having the RTIPS complete the processing of the entire set of marked up danger zones segments to identify those with dangerous embolization agent flows on the workstation screen with appropriate visual/audible alarms.
ii. exit processing when the first dangerous embolization agent flow is identified.

6. The accuracy improved system of claim 5, further comprised of operative linkage to at least a local and a cloud-based hub, controlled by user via at least the programmable key interface.

7. The accuracy improved system of claim 6, further comprised of at least an initial reference scan being image color subtracted from (registered with) live scan stream color data.

8. The accuracy improved system of claim 7, further comprised of software-being configured to interface with users, hubs and data sources as a Real-time Image processing algorithm system (RTIPS).

9. A Real-time Image processing algorithm system (RTIPS) of claim 8, defined by being processed once per frame of the live x-ray scanner data stream with the time available for each processing cycle is determined by the data stream frame rate using dedicated graphics processing and graphics memory and high CPU clock speeds is to detect dangerous embolization flows at high speed and with high reliability by minimizing the number of image pixels that participate in the search process.

10. The Real-time Image processing algorithm system (RTIPS) of claim 8, comprised of further refinements wherein the use of danger zone mark-up tools to pre-determine vulnerable locations in the image frame limits the number of pixels to be processed to a very small fraction of the full frame and yields a high probability of reliable detection of dangerous embolization agent flow within the processing time available.

11. The accuracy improved system of claim 8, used to address issues in disease states/regions/procedures comprised of: Middle Meningeal Artery Embolization; along with analogous data set harvesting during Embolization of arterio-venous malformations of the brain; Embolization of arterio-venous fistulae of the brain and its coverings (dura); Cerebral Aneurysm embolization; Neuro-interventional embolization procedures of the head, neck and central nervous System; Injection of cement into bony structures (during kyphoplasty or vertebroplasty for example); Hardware (screw) placement during spinal instrumentation procedures; Arterial embolization procedures of organs or lesions in the human body; and, Venous embolization procedures of organs or lesion in the human body.

12. The Real-time Image processing algorithm system (RTIPS) of claim 9, comprised of AI interfacing controlled by users/clinicians, namely at least a hub master driving local operating theatres harvesting an and integrating data including AI data, whereby the Cloud-based machine learning hub collects operational data from all local participating operating theatres on local regional and super-regional scale, including storage of all participating operating theatre procedure data managing the global machine learning and AI database.

13. The Real-time Image processing algorithm system (RTIPS) of claim 12, further comprised of storage of current real-time machine learning and AI; optimized program code for local nodes and managed distribution of program code updates to local and participating operating theatres.

14. The Real-time Image processing algorithm system (RTIPS) of claim 13 further comprised of: Danger zone segments, Danger zone segment pixels, Image processing cursor search zone pixels and Image processing cursor pixels defined by a user in a layer showing where the liquid embolism agent is not permitted to flow safely.

15. The Real-time Image processing algorithm (RTIP) of claim 13, wherein as commanded the loop iterates on Danger zone segments; Danger zone segment pixels; Image processing cursor search zone pixels and Image processing cursor pixels driving definitional boundary zones and outputs from impinging on the same.

16. A method of offering for sale, alone or bundled with other devices, real-time Image processing algorithm system by combining hardware and software elements to define surgical boundary conditions, such as embolization danger zones, to facilitate better management of endovascular procedures, inter alia, comprised of, in combination: an x-ray scanner; a computer workstation (desktop/laptop); a programmable key interface; an orthogonal crosshair image processing cursor set; hardware and software with on-line analytical processing (OLAP) functionality and a real-time Image processing algorithm system (RTIPS) with cloud-based and local hub linkages.
